SEQUENCE LISTING (13 sequences)

SEQ ID NO: 1 - RNA, 21 nt, synthetic construct
  source 1..21: mol_type = other RNA
agggagggacgggggctgtgc

SEQ ID NO: 2 - DNA, 89 bp, synthetic construct
  source 1..89: mol_type = other DNA
gccggcgcccgagctctggctccgtgtcttcactcccgtgcttgtccgaggagggagggagggacgggggctgtgctggggcagctgga

SEQ ID NO: 3 - DNA, 50 bp, synthetic construct
  source 1..50: mol_type = other DNA
gtgcagggtccgaggtcagagcgacgtgcgcaattttttttttttttttt

SEQ ID NO: 4 - DNA, 20 bp, synthetic construct
  source 1..20: mol_type = other DNA
cgcggagggagggacggggg

SEQ ID NO: 5 - DNA, 18 bp, synthetic construct
  source 1..18: mol_type = other DNA
acgtgcagggtccgaggt

SEQ ID NO: 6 - DNA, 19 bp, synthetic construct
  source 1..19: mol_type = other DNA
ctgactccgagaaggcgtc

SEQ ID NO: 7 - DNA, 22 bp, synthetic construct
  source 1..22: mol_type = other DNA
gtattcaccacgtttgtggagc

SEQ ID NO: 8 - DNA, 22 bp, synthetic construct
  source 1..22: mol_type = other DNA
actggacatcacagcagaatga

SEQ ID NO: 9 - DNA, 20 bp, synthetic construct
  source 1..20: mol_type = other DNA
cttcgtgaccagctgtcctt

SEQ ID NO: 10 - DNA, 20 bp, synthetic construct
  source 1..20: mol_type = other DNA
gcagccaggagacaagaact

SEQ ID NO: 11 - DNA, 20 bp, synthetic construct
  source 1..20: mol_type = other DNA
agcgcttcactctttcacga

SEQ ID NO: 12 - DNA, 22 bp, synthetic construct
  source 1..22: mol_type = other DNA
ttgttacaggaagtcccttgcc

SEQ ID NO: 13 - DNA, 22 bp, synthetic construct
  source 1..22: mol_type = other DNA
atgctatcacctcccctgtgtg

(19) United States
(12) Patent Application Publication
(10) Pub. No.: US 2023/0088599 A1
(43) Pub. Date: Mar. 23, 2023

(54) MIR-149-3P AND METHOD FOR TREATING METABOLIC DISEASE USING THE SAME

(71) Applicant: Henan University, Kaifeng (CN)

(72) Inventors: Weidong CHEN, Kaifeng (CN); Xiaobo NIE, Kaifeng (CN); Yandong WANG, Beijing (CN); Yun ZHOU, Kaifeng (CN); Xinrui LYU, Kaifeng (CN)

(21) Appl. No.: 17/823,076

(22) Filed: Aug. 29, 2022

Related U.S. Application Data
(63) Continuation-in-part of application No. 16/264,585, filed on Jan. 31, 2019, now abandoned.
Application No. 16/264,585 is a continuation-in-part of application No. PCT/CN2017/081994, filed on Apr. 26, 2017.

(30) Foreign Application Priority Data
Aug. 11, 2016 (CN) 201610655996.9

(51) Int. Cl. C12N 15/113; A61P 3/10; A61P 1/16; A61P 9/10; C12N 15/86

(52) U.S. Cl. CPC C12N 15/113; A61P 3/10; A61P 1/16; A61P 9/10; C12N 15/86; C12N 2310/141; C12N 2310/3515; C12N 2710/10343

(57) ABSTRACT

MicroRNA, including one of or a combination of the following components: (a) a pri-miRNA of miR-149-3p; (b) a pre-miRNA of miR-149-3p; (c) a mature miRNA of miR-149-3p; (d) a miR-149-3p derivative; (e) a 18-26 nucleotides miRNA having a sequence of 5′-AGGGAGG-3′; and (f) a derivative of the 18-26 nucleotides miRNA of (e). Also provided is a method for treating a metabolic disease. The method includes preparing a pharmaceutical composition comprising a mimic of miR-149-3p, or an expression vector of the miR-149-3p, and administering an effective amount of the pharmaceutical composition to a patient in need thereof.

### CROSS-REFERENCE TO RELATED APPLICATIONS

This application is a continuation-in-part of U.S. patent application Ser. No. 16/264,585, filed on Jan. 31, 2019, which is now pending and a continuation-in-part of International Patent Application No. PCT/CN2017/081994 with an international filing date of Apr. 26, 2017, which is now abandoned as to the United States and is based on Chinese Patent Application No. 201610655996.9 filed Aug. 11, 2016. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### INCORPORATION-BY-REFERENCE OF SEQUENCE LISTING

This application contains a sequence listing, which has been submitted electronically in XML file and is incorporated herein by reference in its entirety. The XML file, created on Aug. 27, 2022, is named ZZLK-01102-UUS.xml, and is 12,562 bytes in size.

### BACKGROUND

This disclosure relates to microRNA and a method for treating a metabolic disease using the same.

MicroRNA (abbreviated miRNA) is a small non-coding RNA molecule containing about 22 nucleotides that functions in RNA silencing and post-transcriptional regulation of gene expression. For example, microRNA-122, microRNA-370 and microRNA-378/378* are post-transcriptional regulators of lipid metabolism; microRNA-33a and microRNA-33b are involved in the regulation of cholesterol and lipid metabolism; microRNA-130a, microRNA-200 and microRNA-410 are involved in the regulation of insulin secretion.

### SUMMARY

The disclosure provides microRNA and a method for treating a metabolic disease using the same.

Provided is microRNA, comprising one of or a combination of the following:
(a) a pri-miRNA of miR-149-3p;
(b) a pre-miRNA of miR-149-3p;
(c) a mature miRNA of miR-149-3p;
(d) a miR-149-3p derivative;
(e) a 18-26 nucleotides miRNA comprising a sequence of 5′-AGGGAGG-3′; and
(f) a derivative of the18-26 nucleotides miRNA of (e).

In one embodiment of the invention, the derivative in (d) and/or (f) is a cholesterol modifier, a locked nucleic acid modifier, a nucleotide modifier, a glycosylation modifier, a hydrocarbon modifier, a nucleic acid modifier, or a combination thereof.

In one embodiment of the invention, in (e), the sequence of 5′-AGGGAGG-3′ is located in positions 2-8 of the miRNA; and the 18-26 nucleotides miRNA comprises more than 50% of activities of the miR-149-3p.

In one embodiment of the invention, the mature miRNA of miR-149-3p comprises a RNA sequence represented by SEQ ID NO: 1, or a derivative thereof, and a DNA sequence encoding the mature miRNA is represented by SEQ ID NO: 2, or a derivative thereof.

Also provided is a method for treating a metabolic disease, the method comprising preparing a pharmaceutical composition comprising a mimic of miR-149-3p, or an expression vector of the miR-149-3p, and administering an effective amount of the pharmaceutical composition to a patient in need thereof. In the method, the mimic of miR-149-3p or the expression vector of the miR-149-3p is the active pharmaceutical ingredient.

In one embodiment of the invention, the metabolic disease comprises obesity, fatty liver, hyperlipidemia, hyperuricemia, hypertension, diabetes, atherosclerosis, stroke, or symptoms thereof. Preferably, the metabolic disease is obesity, fatty liver, hyperlipidemia, diabetes, or atherosclerosis. In one embodiment of the invention, the diabetes is type 2 diabetes.

In one embodiment of the invention, the method further comprises integrating a DNA sequence encoding a mature miRNA of miR-149-3p to a viral vector or a eukaryotic vector to construct the expression vector of the miR-149-3p. In one embodiment of the invention, the viral expression vector comprises an adenovirus vector, an adeno-associated virus vector, a retroviral vector, a herpes virus vector, or a combination thereof; and the eukaryotic expression vector comprises PCMV-myc expression vector, pcDNA3.0, pcDNA3.1, a modifier thereof, or a combination thereof.

In one embodiment of the invention, the pharmaceutical composition further comprises a promoter, or an enhancer. The pharmaceutical composition is in the form of a granule, a sustained-release agent, a microinjection, a transfectant, a surfactant, or a combination thereof.

In one embodiment of the invention, the pharmaceutical composition comprising the expression vector of the miR-149-3p is introduced or transfected into the patient's cells or allogeneic cells in vitro, and the cells are amplified in vitro and then transferred to the patient.

In one embodiment of the invention, the pharmaceutical composition comprising the expression vector of the miR-149-3p is directly introduced to the patient.

Also provided is a method of diagnosis of type 2 diabetes, comprising:
1) extracting total microRNAs of claim 1 from a patient's blood and preparing corresponding cDNAs thereof;
2) measuring an expression level of mature microRNAs by fluorescence quantitative PCR; and
3) evaluating the mature microRNAs.

In one embodiment of the invention, a reverse transcription primer as shown in SEQ ID NO: 3 is employed to prepare the corresponding cDNAs.

In one embodiment of the invention, the fluorescence quantitative PCR comprises dye detection and/or probe detection.

In one embodiment of the invention, the fluorescence quantitative PCR employs a forward primer as shown in SEQ ID NO: 4, and a reverse primer as shown in SEQ ID NO: 5.

Advantages of the microRNA and the use thereof for treating a metabolic disease according to embodiments of the disclosure are summarized as follows. The microRNA can improve the insulin sensitivity, reduce the abnormal accumulation of triglycerides in liver, and reduce the deposition of lipid plaques in blood vessels, the blood glucose level, and blood lipid level, thus inhibiting the occurrence and development of metabolic diseases. The microRNA can be used to prepare drugs for the prevention and treatment of metabolic diseases and for the diagnosis and treatment of metabolic diseases. The microRNA can also be used as an auxiliary detection means for the diagnosis of type 2 diabetes.

### BRIEF DESCRIPTION OF THE DRAWINGS

FIG. 1 is fluorescence quantitative PCR results of miRNA mimics-induced expression of miR-149-3p in mouse hepatocarcinoma cells as described in the disclosure;

FIG. 2 is fluorescence quantitative PCR results of expression of miR-149-3p in mouse hepatocarcinoma cells as described in the disclosure;

FIG. 3 is fluorescence quantitative PCR results of miRNA mimics-induced expression of miR-149-3p in obese mice as described in the disclosure;

FIG. 4 is triglyceride levels in the liver measured using a triglyceride test kit;

FIG. 5 is a comparison of staining results of liver and aortic arch in mice;

FIG. 6 is a comparison of relative expression levels of mature miRNA in type 2 diabetes group and healthy control group;

FIG. 7 is correlation analysis diagram of relative expression level of mature miRNA and fasting blood glucose level in detected samples.

FIG. 8 depicts expression levels of miR-149-3p in peripheral blood from healthy people (n=30) and patients with T2DM (n=30). PCR test results indicate that miR-149-3p is expressed at a lower level in patients with T2DM than in healthy people. U6 rRNA is used as a reference gene to normalize the expression of miR-149-3p;

FIGS. 9A-9B depict a negative correlation between miR-149-3p expression levels in peripheral blood from patients with T2DM and levels of both body mass index (BMI) and fasting blood glucose (FBG), respectively;

FIGS. 10A-10B depict a decrease in expression levels of miR-149-3p in cells with insulin resistance induced by sodium oleate and palmitic acid, respectively. HepG2 and Hepa1-6 cells are treated with sodium oleate (100 μM) for 24 hours; and the expression levels of miR-149-3p in HepG2 and Hepa1-6 cells are measured using quantitative real-time PCR;

FIG. 11 depicts a decrease in expression levels of miR-149-3p in the liver of mice with T2DM (n=5). A group of mice is fed a high-fat diet and injected intraperitoneally with streptozotocin (STZ); the other group of mice (WT, n=5) fed common chow is used as a control; the two groups of mice are anesthetized and sacrificed 30 days after STZ injection, and liver tissues are collected; the expression levels of miR-149-3p in the liver tissues are measured using quantitative real-time PCR;

FIGS. 12A-12C: miR-149-3p knockout (miR-149-3p-KO) mice (n=5) and wild-type mice (WT, n=5) are fed a high-fat diet for 12 weeks; blood is collected from the mice and tested through a glucose tolerance test (GTT), an insulin tolerance test (ITT), and a pyruvate tolerance test (PTT). (A) GTT results show that miR-149-3p deletion reduces glucose tolerance of the mice with insulin resistance. (B) ITT results show that miR-149-3p deletion reduces insulin sensitivity of the mice with insulin resistance. (C) PTT results show that miR-149-3p deletion increases hepatic gluconeogenesis in the mice with insulin resistance;

FIG. 13 depicts the effect of miR-149-3p deletion on blood lipid levels in the mice with insulin resistance. When compared to the wild-type mice (n=5), the miR-149-3p-KO mice (n=5) have higher TG and LDL-C levels, a comparable TC level, and lower HDL-C level;

FIGS. 14A-14B depict the effect of miR-149-3p deletion on plasma insulin level and Homeostasis model assessment-estimated insulin resistance (HOMA-IR). Plasma insulin (A) and HOMA-IR (B) levels are higher in the plasma of the miR-149-3p-KO mice (n=5) compared to the wild-type mice (n=5);

FIG. 15 depicts the histopathological results (200×, Scale bar: 100 μm) of the liver tissues in the wild-type mice with insulin resistance mice and the miR-149-3p-KO mice. Steatosis is observed in the liver tissues of the wild-type mice with insulin resistance mice, particularly the miR-149-3p-KO mice (with greater the number of lipid droplet);

FIGS. 16A-16B depict the effect of miR-149-3p deletion on blood glucose level and insulin sensitivity in the mice with T2DM. (A) The wild-type mice with T2DM (n=5) and the miR-149-3p-KO mice with T2DM (n=5) are injected with streptozotocin (STZ), fed for 15 days and fasted for 6 hours. When compared with the wild-type mice with T2DM, the miR-149-3p-KO mice with T2DM have higher blood glucose and the random blood glucose levels, as well as higher random blood glucose levels 22 days after STZ injection. (B) ITT results show that miR-149-3p deletion reduces insulin sensitivity of the mice with T2DM;

FIG. 17 depicts the effect of miR-149-3p deletion on blood lipid levels in the mice with T2DM. When compared to the wild-type mice (n=5), the miR-149-3p-KO mice (n=5) have a higher LDL-C level, a comparable TG and TC levels, and a lower HDL-C level in the plasma;

FIGS. 18A-18B depict the effect of miR-149-3p deletion on levels of plasma insulin and homeostasis model assessment-estimated insulin resistance (HOMA-IR). Plasma insulin (A) and HOMA-IR (B) levels are higher in the miR-149-3p-KO mice (n=5) compared to the wild-type mice (n=5);

FIGS. 19A-19C: the wild-type mice are fed a high-fat diet for 8 weeks and are randomly assigned to two groups of five mice each. The wild-type mice are injected via tail veins with adenovirus vector overexpressing miR-149-3p (miR-149-3p-pAdeno-micro-GFP Adenovirus, abm, Canada) or control adenovirus (1×10⁹ cfu per mice). (A) GTT results show that miR-149-3p overexpression improves the glucose tolerance of the mice with insulin resistance. (B) ITT results show that miR-149-3p overexpression improves the insulin sensitivity of the mice with insulin resistance. (C) PTT results show that miR-149-3p overexpression increases hepatic gluconeogenesis in the mice with insulin resistance;

FIG. 20 depicts decreases in blood glucose levels in the overexpression miR-149-3p mice on day 4 (16-hour fasting), day 6 (6-hour fasting), and day 8 (16-hour fasting) after injection of adenovirus;

FIGS. 21A-21B depict (A) an increase in expression levels of miR-149-3p in the liver tissues of miR-149-3p overexpression mice. The mice are injected with adenovirus, fed for 10 days, anesthetized and sacrificed; and liver tissues are collected and tested for expression level of miR-149-3p by quantitative real-time PCR. (B) The activities of plasma alanine aminotransferase (ALT) and aspartate aminotransferase (AST) between the two groups of the mice are not significantly different;

FIG. 22 depicts the effect of miR-149-3p overexpression on blood lipid levels in the mice with insulin resistance. When compared to the wild-type mice (n=5), the miR-149-3p-KO mice (n=5) have a lower LDL-C level, comparable TC and TG levels, and a higher HDL-C level in the plasma;

FIGS. 23A-23B depicts the effect of miR-149-3p overexpression on plasma insulin level and homeostasis model assessment-estimated insulin resistance (HOMA-IR). Plasma insulin (A) and HOMA-IR index (B) levels are lower in the miR-149-3p-KO mice (n=5) compared to the wild-type mice (n=5);

FIG. 24: miR-149-3p-KO overexpression activates insulin signaling pathway. When hepa1-6 cells are infected with adenovirus overexpressing miR-149-3p and control adenovirus for 48 hours, phosphorylation levels of p-AKT and p-GSK3β in the miR-149-3p overexpression cells are higher than in the control group, according to Western Blot analysis. GAPDH is used as an internal reference protein.

### DETAILED DESCRIPTION

To further illustrate, embodiments detailing microRNA are described below. It should be noted that the following embodiments are intended to describe and not to limit the disclosure.

Unless otherwise mentioned, the “miR-149-3p” mentioned in the disclosure includes a pri-miRNA of miR-149-3p, a pre-miRNA of miR-149-3p, a mature miRNA of miR-149-3p, or a modifier or derivative thereof.

The term “processing” used in the disclosure refers to the entire biological process of obtaining mature miRNA from DNA. In the eukaryotic cells, the process can be completed automatically and generate a primary miRNA (pri-miRNA), a precursor miRNA (pre-miRNA) and a mature miRNA. The DNA is not limited to the source, and includes but is not limited to chromosome DNA and vector DNA.

The operations in the following examples are normal operations unless otherwise specified.

### Materials

Mouse hepatocarcinoma cell line Hepa1-6: Wuhan BOSTER Biological Technology Co., Ltd.

C57BL/6J mouse: Beijing Vital River Laboratory Animal Technology Co., Ltd.

Tri reagent: Jiangsu Enmo Asai Biotechnology Co., Ltd.

Nuclease-Free Water: Ambion, Inc. U.S.A.

Cholesterol modified miR-149-3p mimics and Cholesterol modified miRNA control: Guangzhou RiboBio Co., Ltd.

Liposome 2000: Invitrogen, Inc., U.S.A.

BioTeke MicroRNA Gene First Chain Synthesis Kit: Beijing Bioteke Corporation

High fat diet (60% kcal Fat): Research Diets, Inc.

Triglyceride (TG enzyme) test kit: Nanjing Jiangcheng Bioengineering Institute

Oil-Red-0 Staining Solution: Nanjing Jiangcheng Bioengineering Institute

2×SYBR Green qPCR Mixture: Applied Biosystems

Primer: Sangon Biotech (Shanghai) Co., Ltd.

RNase-free ddH2O: Ambion, Inc. U.S.A.

miRNA cDNA First Chain Synthesis Kit: Beijing Bioteke Corporation

Unless otherwise specified, the reagents used in the disclosure may be any appropriate commercial reagent; cell lines can be obtained from the market. MiRNA mimics in the following examples are cholesterol modified miRNA with greater stability and longer time-effect in cells.

### EXAMPLE 1

### Effect of miR-149-3p on Insulin Signaling Pathway

### 1. Cell Culture

Mouse hepatocarcinoma cell line Hepa1-6 was cultured in DMEM medium (Thermo, USA). The medium contained 10% fetal bovine serum (Gibco, USA) and penicillin-streptomycin solution (100×). All cells were cultured in a 37° C. incubator with 5% CO₂.

### 2. Cell Transfection

The Hepa1-6 cells were plated for 20 hours with the density per well about 60%. Thereafter, a miR-149-3p group and a miRNA negative control group were provided for cell transfection. The transfection reagent used was liposome 2000. The transfection method was carried out according to the instructions.

### 3. Extraction of RNA

After transfection, the cells were cultured for 48 hours and collected. 0.5 mL Tri reagent was added to the cells of each well at room temperature. 5 min later, the bromocresol purple (BCP) solution with 1/10 of the volume of the Tri reagent was added, mixed for 15 seconds and then left at room temperature for 10 min. The mixture was centrifuged under the centrifugal force of 13400 g at 4° C. for 15 min. The supernatant was transferred to a new 1.5 mL centrifugal tube. Isopropanol with equal volume of the supernatant was added. After several times of mixing, the mixture was left alone at room temperature for 10 min, centrifuged under the centrifugal force of 13400 g at 4° C. for 10 min. The supernatant was removed, and 500 μL of 75% ethanol solution (freshly prepared with RNase-free water) was added to clean the RNA. Thereafter, the RNA was centrifuged and precipitated under the centrifugal force of 13400 g at 4° C. for 5 min. The supernatant was removed, and the RNA was dried at room temperature for 5 min. Appropriate nuclease-free water was added to the RNA and the mixture was placed in a 55° C. water bath for 10 min for full dissolution. The absorption values of OD260 and OD280 were determined. It is believed that the A260/A280 of between 1.8 and 2.1 means the total RNA is qualified.

### 4. Detection of the Expression of miR-149-3p and Insulin Signaling pathway-related genes by fluorescence quantitative PCR

2 μg of RNA was employed as a template. Poly (A) tail was added to miRNA using a miRNA cDNA first strand synthesis kit. The resulting miRNA was reversely transcribed to yield cDNA. The cDNA was employed as a template, and amplified using ABI 7500 fluorescence quantitative PCR instrument in the presence of miR-149-3p primers and PCR 2×SYBR Green qPCR Mixture. The PCR parameters were: 50° C. for 20 seconds; 95° C. for 10 minutes; 95° C. for 1 minute; 60° C. for 1 minute, repeat 40 cycles. The CT values of the amplification of the sample mir-149-3p were measured, and the CT values of the internal reference gene U6 were standardized for correction. Meanwhile, the expression levels of key genes in the insulin signaling pathway, such as protein kinase B2 (Akt2), insulin receptor substrate-1 (Irs1), insulin receptor substrate-2 (Irs2), were detected and corrected with beta-actin as an internal reference gene. The CT values were calculated by 2^{-ΔΔCT} method, and the differences of gene levels in different treatment groups were compared.

The forward primer of the Akt2 is shown in SEQ ID NO: 6; the reverse primer of the Akt2 is shown in SEQ ID NO: 7. The forward primer of the Irs1 is shown in SEQ ID NO: 8; the reverse primer of the Irs1 is shown in SEQ ID NO: 9. The forward primer of the Irs2 is shown in SEQ ID NO: 10; the reverse primer of the Irs2 is shown in SEQ ID NO: 11. The forward primer of the internal reference gene β-actin is shown in SEQ ID NO: 12; the reverse primer of the internal reference gene β-actin is shown in SEQ ID NO: 13.

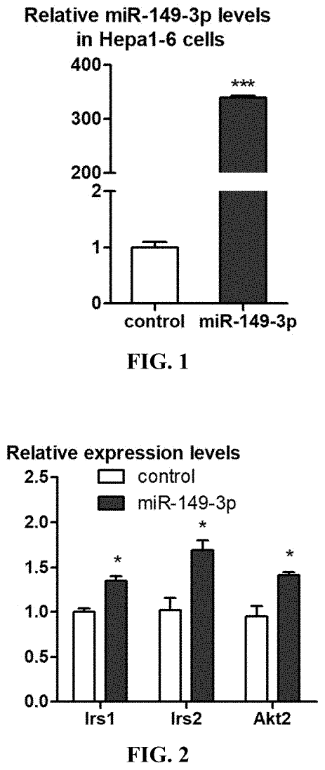

FIG. 1 is fluorescence quantitative PCR results of miRNA mimics-induced expression of miR-149-3p in mouse hepatocarcinoma cells.

FIG. 2 shows that after the expression of the miR-149-3p in mouse liver cancer cells, the expression of genes related to the insulin signaling pathway is increased and the insulin signaling pathway is activated. β-actin was used as internal reference.

Results: Fluorescence quantitative PCR analysis showed that transfection of 200 pmol of mature miRNA can significantly improve the expression level of miR-149-3p in Hepa1-6 cells, as shown in FIG. 2. Meanwhile, the transcription levels of the key genes comprising Akt2, Irs1 and Irs2 of the insulin signaling pathway are all increased to varying degrees. The experiment showed that increasing the expression level of the miR-149-3p by an exogenous method can activate the insulin signaling pathway and improve the insulin resistance of cells.

### EXAMPLE 2

### Effect of Over-Expression of miR-149-3p on Lipid Content in Liver and Vascular Plaque

### 1. Animal Model Construction

Six-week-old male C57BL/6J mice were fed at 22-24° C. in SPF grade animal room. After 12 hours of circadian rhythm and 12 weeks of feeding with high-fat diet, the mice were injected with miR-149-3p mimics (15 mg/kg) or negative control via the tail vein twice. The mice were fed with high-fat diet for 4 consecutive weeks. The mice were anesthetized with ether and killed. Liver and aortic arch were taken. The use and operation of the mice were conducted in strict accordance with the ethics and animal welfare committee.

### 2. Detection of the Expression of miR-149-3p In Vivo by Fluorescence Quantitative PCR

The extraction method of the total RNA from tissues was the same as that from cells, except that 1 mL Tri reagent was added to every 100 mg of tissue, and the tissue blocks were crushed on ice. Following the reverse transcription, the changes of the miR-149-3p levels in tissues were detected by fluorescence quantitative PCR.

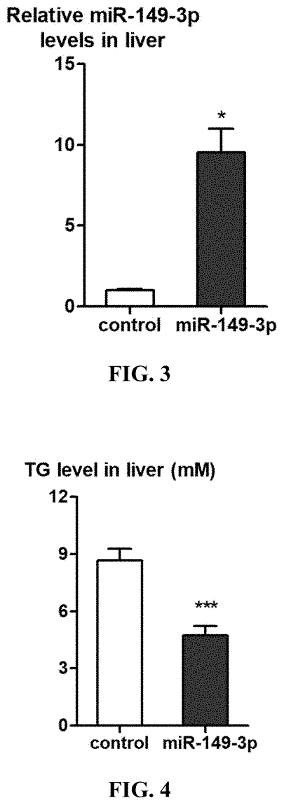

Results: Fluorescence quantitative PCR analysis showed that, as shown in FIG. 3, the expression level of the miR-149-3p molecule in the liver of the mice in the miR-149-3p group (n=4) was significantly increased, nearly 10 times higher than that in the liver of the mice in the control group (n=4). The results showed that injection of exogenous miR-149-3p can ensure the expression of the miR-149-3p in tissues.

### 3. Determination of Fat Content in Mouse Liver

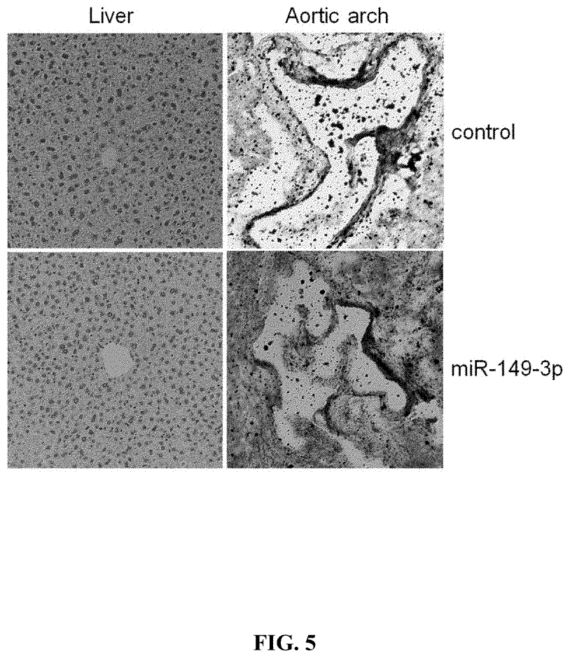

The mouse livers of the two groups were stained with H&E. The liver tissue of the mice fed with high-fat diet showed obvious lipid droplets vacuoles (as shown in FIG. 5), indicating abnormal accumulation of fat in the liver, but the number of lipid droplets vacuoles in the liver decreased significantly after over-expression of the miR-149-3p. Triglyceride levels in the liver were also measured using a triglyceride kit according to the operating instructions. The results showed (as shown in FIG. 4) that expression of miR-149-3p significantly reduced triglyceride levels in the liver.

### 4. Pathological Changes of Aorta in Mice

The mice were anesthetized and killed, and the aortic root was quickly taken for frozen section. The sections were stained with oil red O staining solution. Observe the formation of plaques in different sections, and the images were collected under the microscope. The staining results were shown in FIG. 5. Oil-Red-O stained plaques were observed on the vessel wall of the aortic root of the mice in the negative control group, which was the site of atherosclerosis, while the Oil-Red-O stained plaques in the aortic vessel wall of the mice in the mir-149-3p group were significantly reduced.

Statistical analysis: All data were averaged by three independent repetitive experiments. Standard deviation (SD) was analyzed by GraphPad Prism 5. P<0.05 was considered statistically significant, and *P<0.05; * P<0.01; ***P<0.001.

Insulin signal transduction pathway mainly refers to the activation of the insulin receptor substrate (Irs), the phosphatidylinositol 3 kinase (pi3-k), and the protein kinase B (Akt) after the insulin binds to the receptor on the target cell, thus promoting the storage of substances. Genetic or environmental factors (such as lack of exercise and high-fat diet) can cause abnormal insulin signaling pathway and lead to insulin resistance. Insulin resistance can cause excessive accumulation of triglycerides in the liver, aggravate the degree of insulin resistance, and lead to hyperlipidemia, fatty liver or type 2 diabetes. Insulin resistance can also cause abnormal lipid metabolism and vascular inflammation, and is an independent risk factor for hypertension and atherosclerosis.

The disclosure teaches key miRNAs affecting insulin signaling pathways and lipid metabolism. Experiments have confirmed that expression of miR-149-3p in the mouse liver cells can up-regulate the expression of key genes in the insulin signaling pathway and activate insulin signal transduction. Meanwhile, expression of miR-149-3p in obese mice induced by high-fat diet can significantly reduce the level of triglycerides in the liver and reduce the abnormal accumulation of lipid droplets in the liver. In addition, the deposition of the lipid plaque in the aortic vessel wall was decreased. The results showed that expression of the miR-149-3p in vivo can be a new strategy for the treatment of metabolic diseases, and the miR-149-3p can be a new potential target for the treatment of such diseases.

### EXAMPLE 3

### Application of Mature miRNA in the Diagnosis of Type 2 Diabetes

### 1. Clinical Sample Collection

Since 2015, a large number of peripheral blood samples from patients with type 2 diabetes from Huaihe Hospital affiliated to Henan University and healthy people were collected. The whole process of collection and follow-up experiment conforms to the requirements of medical ethics. Sampling, packing and preservation conditions of the samples are the same. By sorting out the medical records, 30 samples were selected for real-time fluorescence quantitative PCR detection.

Healthy population with fasting blood glucose of between 3.9 and 6.1 mmol/L was defined as a healthy control group.

Population having a fasting blood glucose greater than or equal to 7.0 mmol/L after two consecutive repeated tests was defined as a patient group, which was diagnosed as type 2 diabetes, and the population received no drug treatment.

### 2. Extraction of Total RNA in Blood

To per 200 μL of fresh blood, 600 μL of Tri reagent was added. The mixture was whirlpool oscillated to lyse the blood cells. 5 min later, the bromocresol purple (BCP) solution with 1/10 of the volume of the Tri reagent was added, mixed for 15 seconds and then left at room temperature for 10 min. The mixture was centrifuged under the centrifugal force of 13400 g at 4° C. for 15 min. The supernatant was transferred to a new 1.5 mL centrifugal tube. Isopropanol with equal volume of the supernatant was added. After several times of mixing, the mixture was left alone at −80° C. for an hour, centrifuged under the centrifugal force of 13400 g at 4° C. for an hour. The supernatant was removed, and 500 μL of 75% ethanol solution (freshly prepared with RNase-free water) was added to clean the RNA. Thereafter, the RNA was centrifuged and precipitated under the centrifugal force of 13400 g at 4° C. for 5 min. The supernatant was removed, and the RNA was dried at room temperature for 5 min. Appropriate nuclease-free water was added to the RNA and the mixture was placed in a 55° C. water bath for 10 min for full dissolution. The absorption values of OD260 and OD280 were determined. It is believed that the A260/A280 of between 1.8 and 2.1 means the total RNA is qualified.

### 3. Detection of Mature miRNA by Fluorescence Quantitative PCR

2 μg of total RNA was employed as a template. Poly (A) tail was added to miRNA using a miRNA cDNA first strand synthesis kit (BioTeke). A reverse transcription system was prepared after the reaction, as shown in Table 1.

TABLE 1

| | |
|---|---|
| Poly(A) reaction solution | 10 μL |
| Reverse transcription primer (10 μM) | 2 μL |
| 5×RT Bufer | 4 μL |
| dNTP (2.5 mM Each) | 1 μL |
| RNase inhibitor (40 U/μL) | 1 μL |
| M-MuLV Reverse transcriptase (200 U/μL) | 0.5 μL |
| RNase-free ddH₂O | 1.5 μL |
| Total volume | 20 μL |

The reverse transcription was carried out at 37° C. for 60 minutes to yield cDNA. The cDNA was diluted to 4 ng/μL as a template for quantitative fluorescence PCR. Amplification was carried out on ABI 7500 fluorescent quantitative PCR instrument in the presence of positive primers of mature microRNAs and internal reference gene U6, general reverse primers and 2*SYBR Green Q PCR Mixture.

The reverse transcription primer of the miRNA as shown in SEQ ID NO: 3; the forward primer is as shown in SEQ ID NO: 4; and the reverse primer is as shown in SEQ ID NO: 5.

The primers for detecting blood miRNA provided in this example are designed based on the poly(A) polymerase tailing method. In certain implementation methods, the real-time fluorescence quantitative PCR primers for detecting the mature miRNA can also be designed according to the stem-loop method. The reaction system was as follows, in which the amplification system of the internal reference gene U6 or mature miRNA was shown in Table 2.

TABLE 2

| | |
|---|---|
| cDNA (20 ng) | 5 μL |
| Forward primer (10 μM) | 0.5 μL |
| General reverse primer (10 μM) | 0.5 μL |
| 2×SYBR Green qPCR Mixture | 10 μL |
| RNase-free ddH₂O | 4 μL |
| Total volume | 20 μL |

The PCR parameters were: 50° C. for 20 seconds; 95° C. for 10 minutes; 95° C. for 1 minute; 60° C. for 1 minute, repeat 40 cycles. The CT values of the amplification of the sample mature miRNA were measured, and the CT values of the internal reference gene U6 were standardized for correction.

### 4. Data Processing and Analysis

The ratio of the microRNAs expression in two groups of blood samples was calculated by 2^{-ΔΔCT} method. ΔΔCT=(CT1(miRNA)-CT1(U6)) -(CT2(miRNA)-CT2(U6)). CTmiRNA is the CT value of amplification of mature miRNA, CTU6 is the CT value of amplification of the internal reference gene U6, CT1 is the CT value of amplification of the patient group or healthy control group, and CT2 is the CT value of amplification in the healthy control group.

TABLE 3

| Sample No. | Type 2 diabetes | Expression level of mature miRNA |
|---|---|---|
| 1 | 0 | 0.97 |
| 2 | 0 | 0.92 |
| 3 | 0 | 1.16 |
| 4 | 0 | 1.82 |
| 5 | 0 | 1.72 |
| 6 | 0 | 0.89 |
| 7 | 0 | 1.26 |
| 8 | 0 | 1.60 |
| 9 | 0 | 1.24 |
| 10 | 0 | 1.13 |
| 11 | 0 | 1.26 |
| 12 | 0 | 1.59 |
| 13 | 0 | 1.43 |
| 14 | 0 | 1.45 |
| 15 | 0 | 0.98 |
| 16 | 1 | 0.77 |
| 17 | 1 | 0.27 |
| 18 | 1 | 0.27 |
| 19 | 1 | 0.73 |
| 20 | 1 | 0.89 |
| 21 | 1 | 0.66 |
| 22 | 1 | 0.51 |
| 23 | 1 | 0.67 |
| 24 | 1 | 0.67 |
| 25 | 1 | 0.68 |
| 26 | 1 | 0.89 |
| 27 | 1 | 0.72 |
| 28 | 1 | 0.83 |
| 29 | 1 | 0.87 |
| 30 | 1 | 0.44 |

In Table 3, “0” represents healthy population, and “1” represents patients with type 2 diabetes.

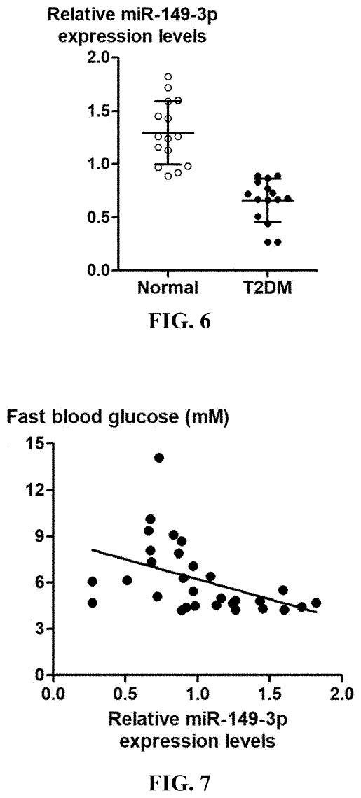

FIG. 6 is a comparison of relative expression levels of mature miRNA in type 2 diabetes group and healthy control group.

FIG. 7 is correlation analysis diagram of relative expression level of mature miRNA and fasting blood glucose level in detected samples.

Statistical analysis by SPSS software showed that the expression levels of mature miRNA in type 2 diabetes patients and healthy control groups were significantly different (P<0.001), and the level of the mature miRNA was significantly negatively correlated with fasting glucose level (R=−0.45, P=0.013), as shown in FIG. 7. P<0.05 was considered statistically significant.

Therefore, it can be concluded that the mature miRNA is significantly decreased in the blood of patients with type 2 diabetes and can be used as a molecular marker for the detection of type 2 diabetes.

The microRNA can improve the insulin sensitivity, reduce the abnormal accumulation of triglycerides in liver, and reduce the deposition of lipid plaques in blood vessels, thus inhibiting the occurrence and development of metabolic diseases. The microRNA can be used to prepare drugs for the prevention and treatment of metabolic diseases and for the diagnosis and treatment of metabolic diseases.

### EXAMPLE 4

### miR-149-3p Being Expressed at Low Levels in Patients with T2DM and Being Negatively Correlated with Indicators of T2DM

Peripheral blood samples were collected from fasting patients with uncomplicated T2DM from Huaihe Hospital affiliated to Henan University and fasting healthy people, and anticoagulated with EDTA. Informed consent must be obtained from research subjects before blood is taken. After reviewing the medical records, 30 peripheral blood samples from patients with uncomplicated T2DM were selected as an experimental group; and 30 peripheral blood samples from healthy people were selected as a control group with matching age and gender. The expression levels of miR-149-3p in the selected peripheral blood samples were measured using quantitative real-time PCR.

### 1. RNA Extraction

0.5 mL of TRI reagent was added to 0.3 mL of anticoagulated blood, mixed on a vortex mixer for 30 seconds, allowed to stand for 5 minutes; a bromocresol purple (BCP) solution with 1/10 of the volumes of TRI reagent was added, mixed on the vortex mixer for 15 seconds, allowed to stand at room temperature for 10 minutes, centrifuged at 4° C. and 13400 g for 15 minutes; the supernatant was transferred into a new 1.5 mL centrifuging tube; isopropanol with an equal volume of the supernatant was added, mixed by inversion several times, allowed to stand at room temperature for 10 minutes, and centrifuged at 4° C. and 13400 g for 10 minutes; the supernatant was removed from the centrifuging tube, leaving only a RNA pellet; 500 μL of 75% ethanol solution (prepared freshly by mixing ethanol with RNase-free water) was added, and the RNA pellet was washed in the 75% ethanol solution and centrifuged at 4° C. and 13400 g for 5 minutes; the supernatant is removed from the centrifuging tube and the RNA pellet was air-dried at room temperature for 5 minutes; nuclease-free water was added and placed in a 55° C. water bath for 10 minutes for complete dissolution of the RNA pellet. The absorption values of OD260 and OD280 were determined. An A260/A280 ratio of between 1.8 and 2.1 was accepted as pure for RNA.

### 2. Detection of miR-149-3p Expression by Quantitative Real-Time PCR

2 μg of RNA was used as a template; a miRNA First Strand cDNA Synthesis Kit (Bioteke, China) was used to add a poly(A) tail to miRNA and reverse transcribes miRNA into cDNA. cDNA was used as a template, mixed with a pair of miR-149-3p primers and a PCR 2×SYBR Green qPCR mixture, and loaded on an ABI 7500 Real-Time PCR machine. PCR cycling and running parameters was set up for miR-149-3p amplification: 50° C. for 20 seconds; 95° C. for 10 minutes; 95° C. for 1 minute; 60° C. for 1 minute, repeat 40 cycles. CT-values for miR-149-3p and a reference gene U6 were calculated by 2-MCT method to normalise miR-149-3p. The expression levels of miR-149-3p in different groups were compared.

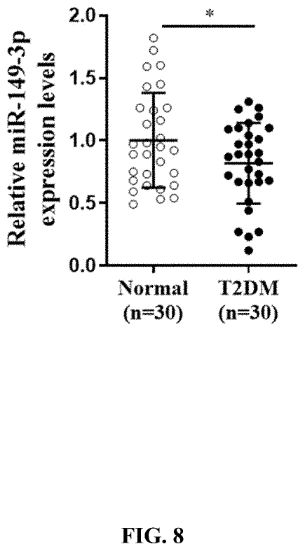

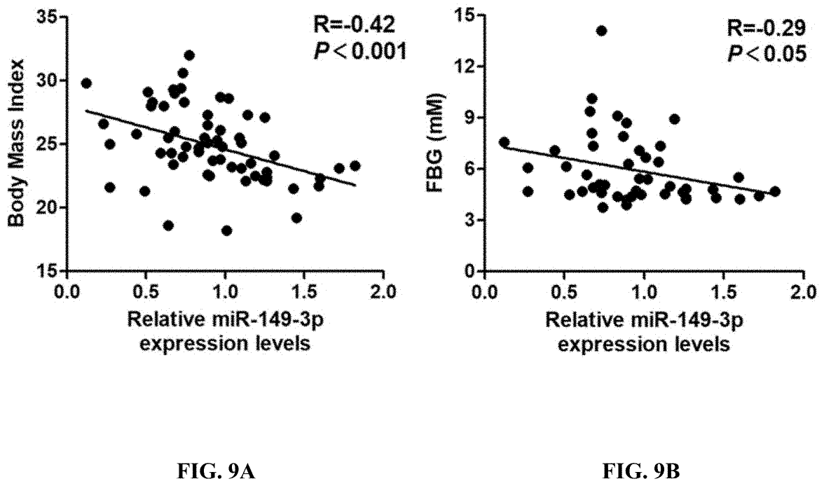

Results: The expression levels of miR-149-3p in the peripheral blood samples from T2DM patients decreased significantly (P<0.05) (as shown in FIG. 8) and were significantly negatively correlated with both body mass index (BMI) and fasting blood glucose (FBG) (as shown in FIGS. 9A-9B). A high BMI was a strong risk factor for insulin resistance and T2DM; and a high FBG pointed to diabetes. The results indicated that a decrease in the expression level of miR-149-3p was closely related to the occurrence of insulin resistance and T2DM, and miR-149-3p may be a protective gene that was associated with decreased risk of T2DM.

### EXAMPLE 5

### Low Expression of miR-149-3p in a Cell-Based Model of Insulin Resistance

Mouse hepatocarcinoma cell line Hepa1-6 and human hepatoma cell line HepG2 were cultured in DMEM complete medium (Thermo, USA). The DMEM complete medium contained 10% fetal bovine serum (Gibco, USA), 100 U/mL penicillin, and streptomycin. Hepa1-6 and HepG2 cells were seeded into a 6-well plate, cultured overnight to a density of about 80%, and then transferred from the DMEM complete medium into a serum-free medium. Sodium oleate or palmitic acid was added to the serum-free medium to achieve a final concentration of 100 μM, thus forming a mouse model of insulin resistance. A control group is treated with a control reagent with the same volume as the serum-free medium. The Hepa1-6 and HepG2 cells were then incubated at 37° C. and 5% CO₂ for 24 hours, collected for total RNA extraction. The expression levels of miR-149-3p in the Hepa1-6 and HepG2 cells were measured using a quantitative Real-Time PCR machine.

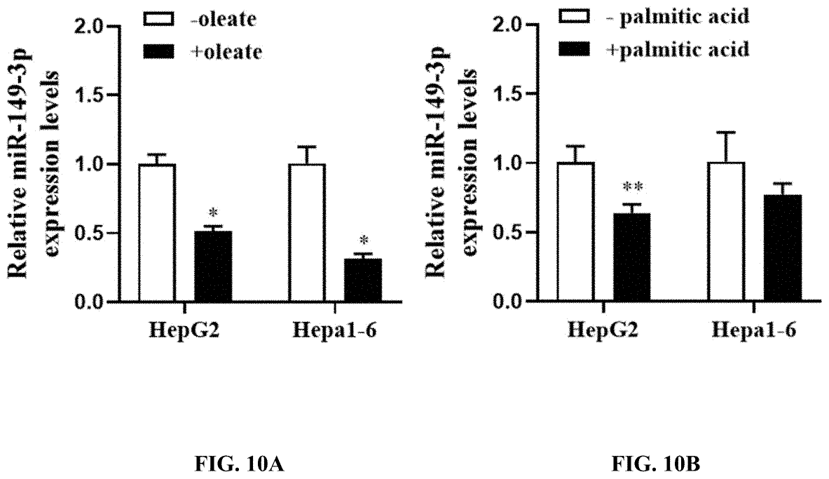

As shown in FIGS. 10A-10B, miR-149-3p was expressed at low levels in the hepatocytes of the mice with insulin resistance.

### EXAMPLE 6

### Low Expression of miR-149-3p in the Liver of Mice with T2DM

To serve as a mouse model of T2DM, four-week-old male C57BL/6J mice (wild-type, WT) and miR-149-3p-KO mice were housed at 22-24° C. in SPF grade animal room, maintained in a 12-hour light/dark cycle, fed a 60% high-fat diet for 8 weeks, and injected with streptozotocin (STZ, 40 mg/kg) once daily for 5 days; ten days later, blood was collected via tail veins on the mice; and the modeling was considered successful when all three random blood glucose levels exceeded 8.3 mmol/L. A group of mice with matching gender and age were fed common chow and used as a control. The mice were anesthetized with ether and sacrificed, and liver tissues were collected for measurement of the expression level of miR-149-3p. The use and operation of the mice were conducted in strict accordance with the ethics and animal welfare committee.

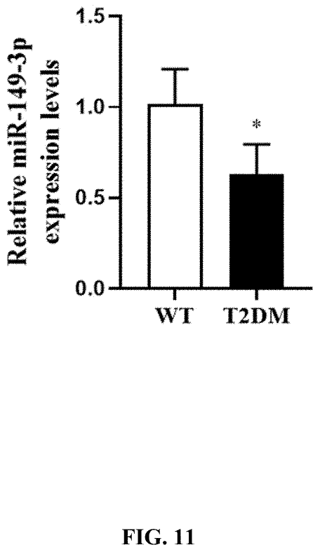

As shown in FIG. 11, the expression level of miR-149-3p was lower in the liver tissues of the mice with T2DM compared to the control group, and it was also low in the T2DM mice as well as the cell-based model of insulin resistance. The results indicate that the decrease of the expression level of miR-149-3p was closely related to the occurrence of insulin resistance and T2DM, and miR-149-3p may be a protective gene that was associated with decreased risk of T2DM.

### EXAMPLE 7

### miR-149-3p Deletion Worsens Insulin Resistance in Mice

### 1. miR-149-3p Deletion Impairs Glucose Metabolism in the Mice with Insulin Resistance

Four-week old, male miR-149-3p knockout (miR-149-3p-KO) mice were used as an experimental group; a group of C57BL/6J (wild-type, WT) mice with matching gender and age was used as a control; the mice were fed a high-fat diet for 12 weeks to serve as a mouse model of insulin resistance. Blood samples were collected via tail veins on the mice and tested for random glucose levels and fasting blood glucose levels.

### 2. Glucose Tolerance Test (GTT)

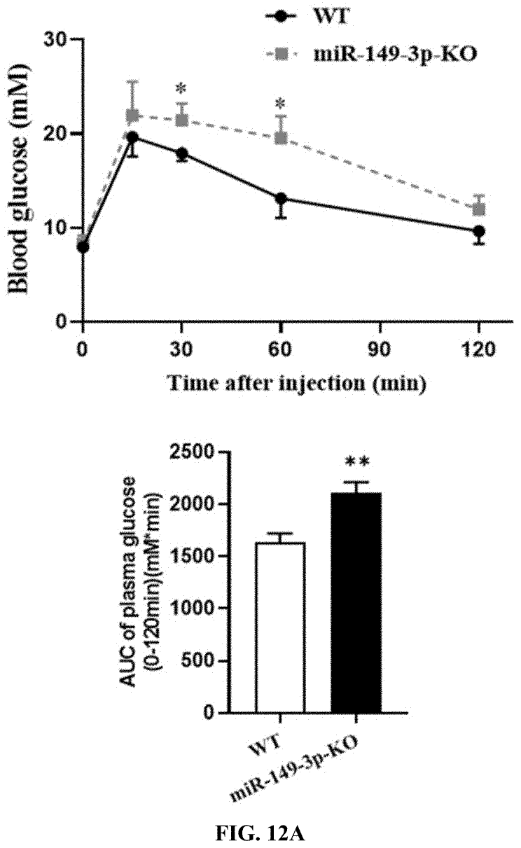

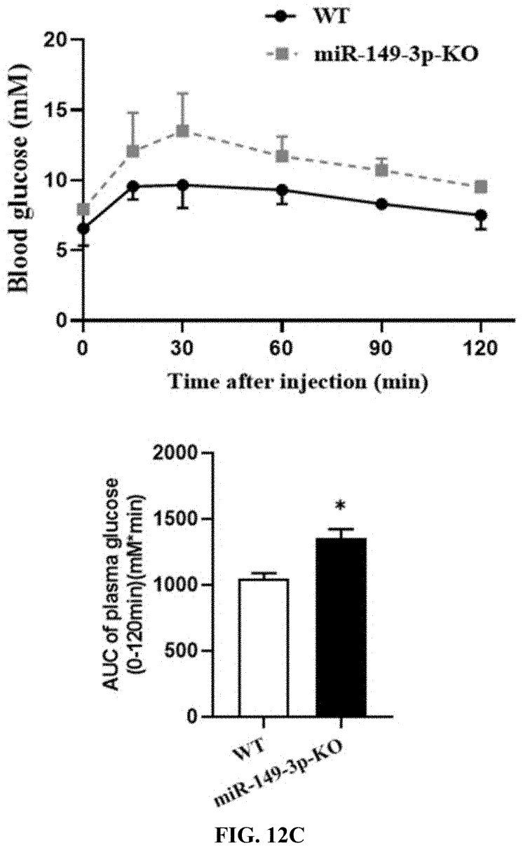

The mice were fasted for one day before glucose tolerance test and weighted 16 hours later. Blood samples were collected via the tail veins and tested for fasting blood glucose levels. The mice were injected intraperitoneally with a 20% glucose solution, at a dose of 2 g/kg (i.e. 10 μL of the 20% glucose solution per 1 g of body weight); blood glucose levels were measured at 15 minutes, 30 minutes, 60 minutes and 120 minutes after injection. A blood glucose curve was drawn according to each blood glucose level at corresponding time point; and the area under the blood glucose curve was calculated. When compared to the wild-type mice, the miR-149-3p-KO mice had higher blood glucose levels at 30 and 60 minutes after injection, and a larger area under the blood glucose curve, indicating that miR-149-3p deletion resulted in a reduction in the glucose tolerance in the mice (FIG. 12A).

### 3. Insulin Tolerance Test (ITT)

The mice were fasted on the day of insulin tolerance test and weighted 6 hours later. Blood samples were collected via the tail veins and tested for fasting blood glucose levels. Mice were injected intraperitoneally with insulin (Novolin-R, Denmark), at a dose of 1.2 U/kg (i.e. 10 μL of insulin per 1 g of body weight); blood glucose levels were measured at 15 minutes, 30 minutes, 45 minutes and 60 minutes after injection. A blood glucose curve was drawn according to each blood glucose level at corresponding time point; and the area under the blood glucose curve was calculated. When compared to the wild-type mice, the miR-149-3p-KO mice had lower blood glucose levels 30 minutes after insulin injection, as well as a larger area under the blood glucose curve, indicating that miR-149-3p deletion resulted in a reduction in the insulin sensitivity of the mice (FIG. 12B).

### 4. Pyruvate Tolerance Test (PTT)

The mice were fasted for one day before pyruvate tolerance test and weighted 16 hours later. Blood samples were collected via the tail veins and tested for fasting blood glucose levels. Mice were injected intraperitoneally with a 20% sodium pyruvate solution (Sigma, USA), at a dose of 2 g/kg (i.e. 10 μL of the 20% sodium pyruvate per 1 g of body weight); blood glucose levels were measured at 15 minutes, 30 minutes, 60 minutes and 120 minutes after injection. A blood glucose curve was drawn according to each blood glucose level at corresponding time point. When compared to the wild-type mice, the miR-149-3p-KO mice had higher blood glucose levels at each time point, as well as a larger area under the blood glucose curve, indicating that miR-149-3p deletion resulted in an increase in the hepatic gluconeogenesis in the mice (FIG. 12C).

### 5. miR-149-3p Deletion Worsens Lipid Disorders in the Mice with Insulin Resistant

The mice were fed a high-fat diet for 14 weeks, fasted for 12 hours, anesthetized and sacrificed. Blood samples were collected from the mice. Plasma was separated from the blood samples and tested for triglycerides (TG) level, total cholesterol (TC) level, low-density lipoprotein cholesterol (LDL-C) level, and high-density lipoprotein cholesterol (HDL-C) level. Kits were purchased from Nanjing Jiancheng Bioengineering Institute, and all operations were performed according to manufacturer's protocol.

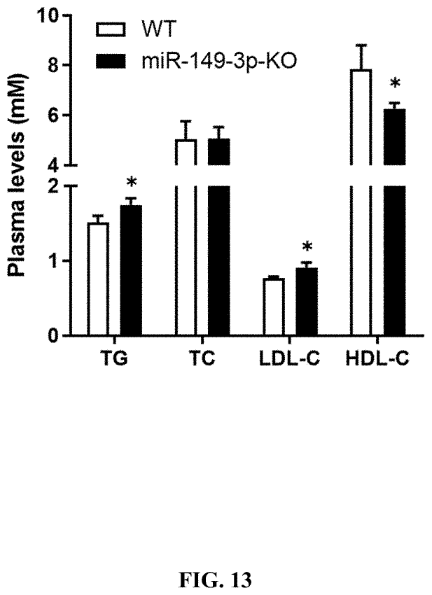

When compared to the wild-type mice, the miR-149-3p-KO mice had higher TG and LDL-C levels, a comparable TC level, and a lower HDL-C level (as shown in FIG. 13). The results showed that miR-149-3p deletion worsened lipid disorders in the mice with insulin resistant.

### 6. miR-149-3p Deletion Increases HOMA-IR

A Mouse Insulin ELISA Kit (Elabscience, China) was used to test the insulin level in the plasma. Homeostasis model assessment-estimated insulin resistance (HOMA-IR) was an important indicator of insulin resistance and calculated according to the formula: fasting blood glucose level (FPG, mmol/L)×fasting insulin level (FINS, μU/mL)/22.5.

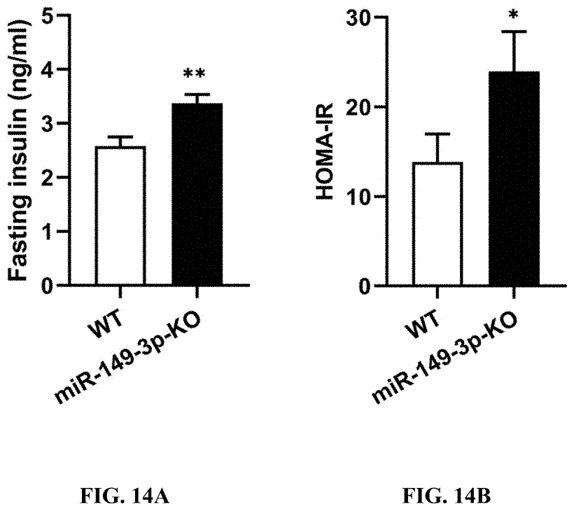

Fasting insulin (FIG. 14A) and HOMA-IR (FIG. 14B) levels are higher in the miR-149-3p-KO mice compared to the wild-type mice, indicating that miR-149-3p deletion worsened insulin resistance in the mice.

### 7. miR-149-3p Deletion Worsens Liver Fatty Disease in Mice

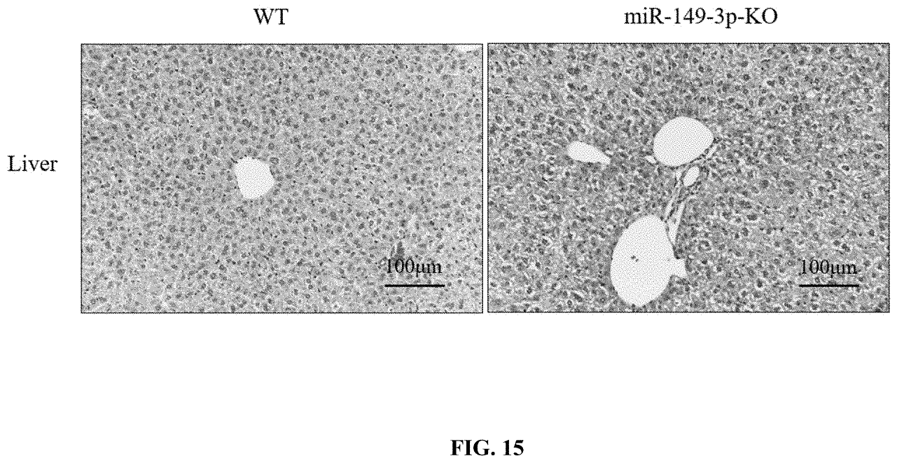

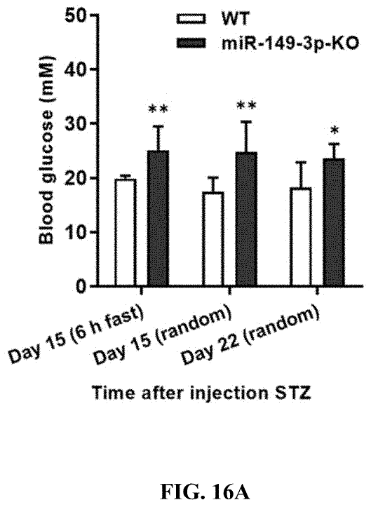

The liver tissues of the wild-type mice and miR-149-3p-KO mice were sectioned and stained by H&E. Steatosis was observed in the liver tissue sections of the wild-type mice, particularly in the miR-149-3p-KO mice, indicating that miR-149-3p deletion aggravated hepatic steatosis in the mice with insulin resistance (FIG. 15).

### EXAMPLE 8

### miR-149-3p Deletion Worsens Diabetic Phenotype in Mice

### 1. miR-149-3p Deletion Aggravates Symptoms of T2DM in Mice.

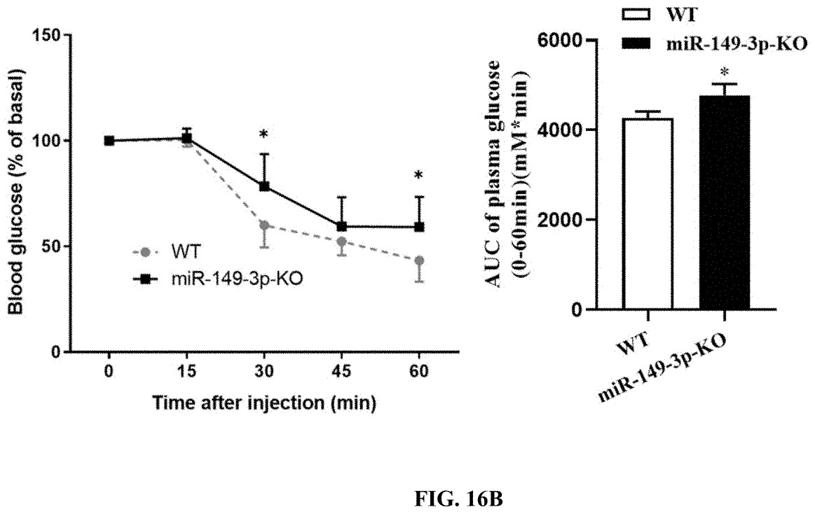

Four-week old, male C57BL/6J (wild-type, WT) mice and miR-149-3p-KO mice were fed a high-fat diet for 8 weeks and injected intraperitoneally with streptozotocin (STZ) to serve as mouse models of T2DM. The fasting blood glucose levels and the random blood glucose levels of the mice with T2DM were measured at different time points. Fasting blood glucose and random blood glucose levels (FIG. 16A) were higher in the miR-149-3p-KO mice with T2DM compared to the wild-type mice with T2DM. Insulin tolerance test (ITT) was carried out 27 days after STZ injection. When compared to the wild-type mice with T2DM, the miR-149-3p-KO mice with T2DM had lower blood glucose levels, as well as a larger area under the blood glucose curve, indicating that miR-149-3p deletion worsened the insulin sensitivity of the mice with T2DM (FIG. 16B).

### 2. miR-149-3p Deletion Worsens Lipid Disorders in the Mice with T2DM

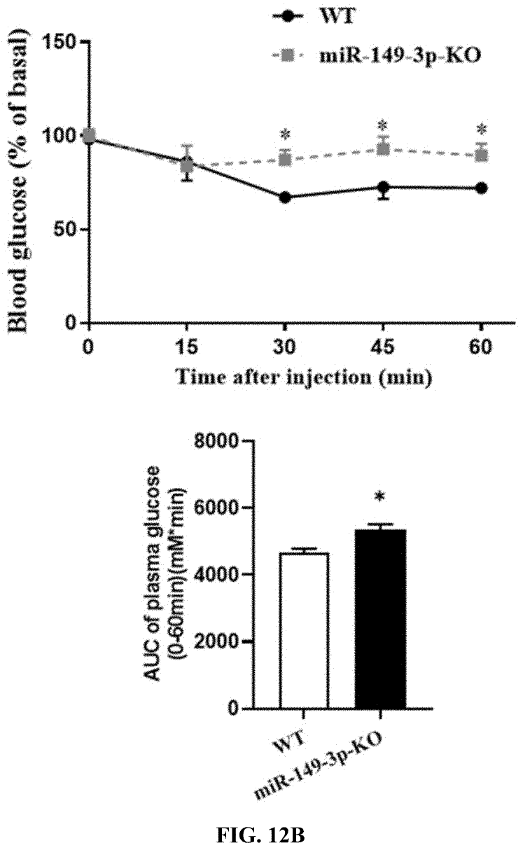

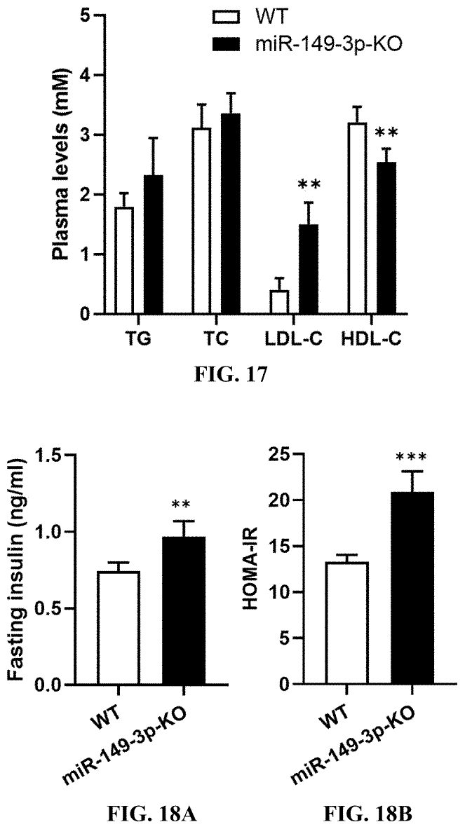

The mice were injected intraperitoneally with STZ, fed for 30 days, fasted for 12 hours, anesthetized for blood sampling, and then sacrificed. Plasma was separated from blood samples and tested for the levels of TG, TC, LDL-C, and HDL-C. As shown in FIG. 17, when compared to the wild-type mice, miR-149-3p-KO mice with T2DM had comparable TG and LC levels, a higher LDL-C level, and a lower HDL-C level. The results indicated that miR-149-3p deletion worsened lipid disorders in the mice with T2DM.

### 3. miR-149-3p Deletion Worsens Insulin Resistance in the Mice with T2DM

A Mouse Insulin ELISA Kit was used to test the insulin level in the plasma. HOMA-IR was calculated from the fasting glucose levels in the mice. The levels of the fasting insulin (FIG. 18A) and HOMA-IR (FIG. 18B) were higher in the miR-149-3p-KO mice with T2D compared to the wild-type mice with T2D, indicating that miR-149-3p deletion worsened insulin resistance in the mice.

### EXAMPLE 9

### miR-149-3p Overexpression Improves Insulin Resistance in Mice

### 1. Construction of Mouse Model

Four-week-old, male C57BL/6J mice (wild-type, WT) were fed a high-fat diet for 8 weeks to serve as mouse models of insulin resistance. The mice were then randomly assigned to two groups and injected via tail veins with adenovirus vector expressing miR-149-3p (miR-149-3p-pAdeno-micro-GFP Adenovirus, abm, Canada) and control adenovirus (1×10⁹ cfu per mice), respectively. The adenovirus vector expressing miR-149-3p (miR-149-3p-pAdeno-micro-GFP Adenovirus, abm, Canada) includes a DNA sequence encoding a mature miRNA of miR-149-3p and the DNA sequence is represented by SEQ ID NO: 2.

### 2. Glucose Tolerance Test (GTT)

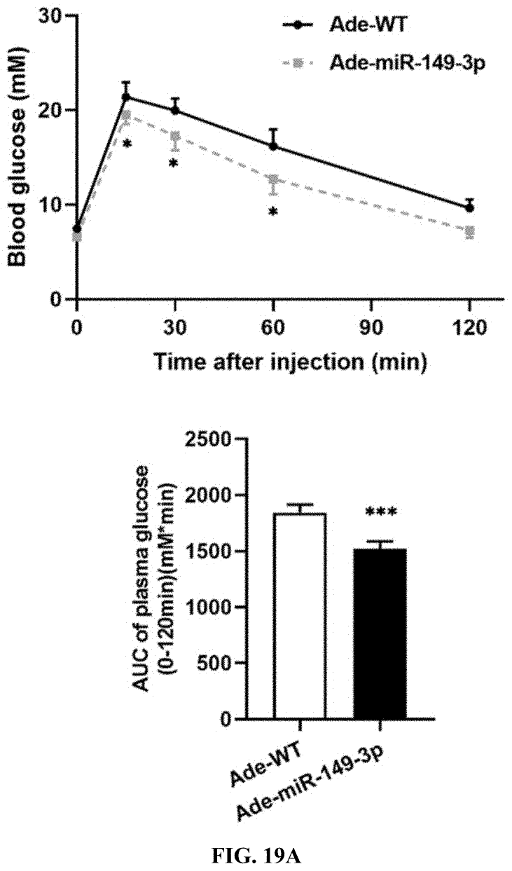

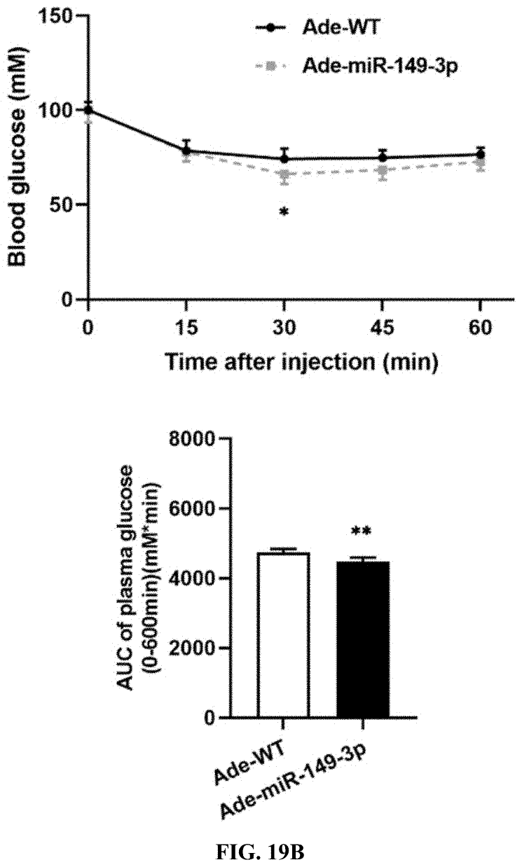

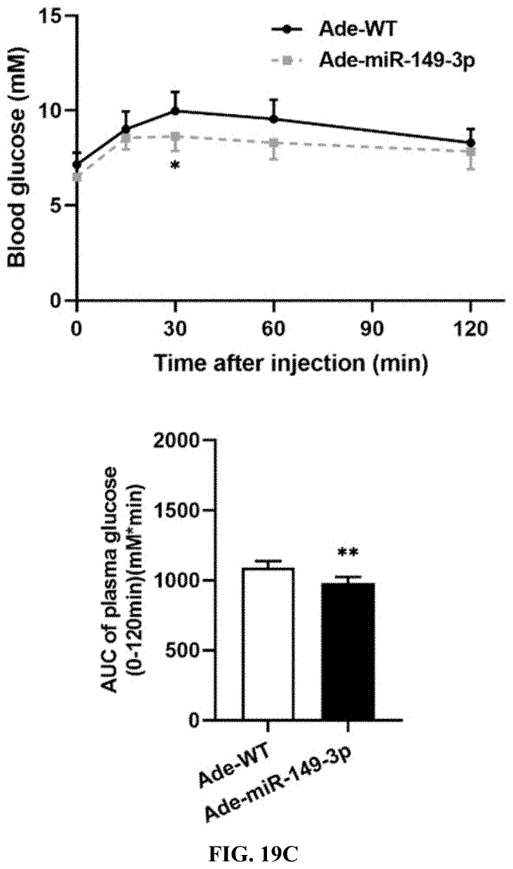

On day 4 after adenovirus injection, the mice were injected intraperitoneally with a glucose solution and tested for the glucose tolerance levels. As shown in FIG. 19A, when compared to the wild-type mice, the miR-149-3p overexpression mice had lower blood glucose levels at 15 minutes, 30 minutes and 60 minutes after injection, as well as a smaller area under the blood glucose curve, indicating that overexpression of miR-149-3p 149-3p improved glucose tolerance in the mice with insulin resistance.

### 3. Insulin Tolerance Test (ITT)

On day 6 after adenovirus injection, the mice were injected intraperitoneally with an insulin solution and tested for the insulin tolerance levels. As shown in FIG. 19B, the blood glucose levels in all mice decreased 30 minutes after injection; when compared to the wild-type mice, the miR-149-3p overexpression mice had higher blood glucose levels, as well as a smaller area under the curve was smaller, indicating that miR-149-3p expression enhances the insulin sensitivity of mice.

### 4. Pyruvate Tolerance Test (PTT)

On day 8 after adenovirus injection, the mice were injected intraperitoneally with a pyruvate solution and tested for the pyruvate tolerance levels. As shown in FIG. 19C, when compared to the control group, the miR-149-3p-KO mice had lower blood glucose levels at 30 minutes and 60 minutes after insulin injection, as well as a smaller area under the blood glucose curve, indicating that miR-149-3p overexpression inhibited hepatic gluconeogenesis in the mice.

### 5. miR-149-3p Overexpression Decreases Blood Glucose Levels in Mice

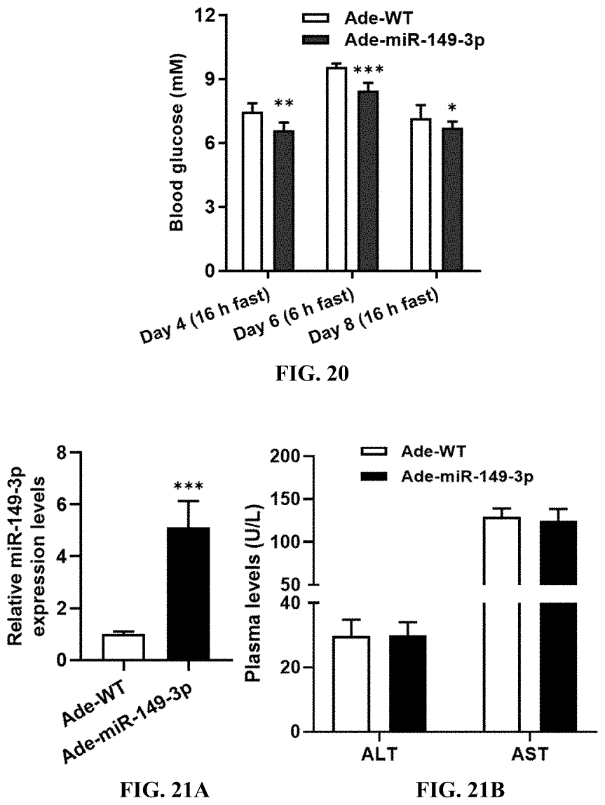

After adenovirus injection, the blood glucose levels were measured on day 4 (16-hour fasting), day 5 (6-hour fasting), day 6 (6-hour fasting), and day 8 (16-hour fasting). As shown in FIG. 20, the blood glucose levels were lower in the miR-149-3p overexpression mice compared to the wild-type mice.

### 6. miR-149-3p expression level increases in the liver tissue of miR-149-3p overexpression mice

On day 10 after adenovirus injection, the mice were fasted for 12 hours, anesthetized for blood sampling, and sacrificed; plasma was separated from the blood samples; liver tissues were collected from the mice; total RNA was extracted from the liver tissues; and the expression levels of miR-149-3p in the liver tissues were measured by quantitative real-time PCR. As shown in FIG. 21A, the expression level of miR-149-3p in the liver tissues of the miR-149-3p overexpression mice (n=5) was five times higher than that in the wild-type mice (n=5), which indicates that the adenovirus as a vector promotes miR-149-3p expression in tissues, resulting in a stable hypoglycemic effect. Meanwhile, miR-149-3p overexpression did not impair the liver function because there were no changes in the activities of alanine transaminase (ALT) and aspartate aminotransferase (AST) in the mice plasma (FIG. 21B).

### 7. miR-149-3p Overexpression Decreases Blood Lipid Levels in the Mice with Insulin Resistant

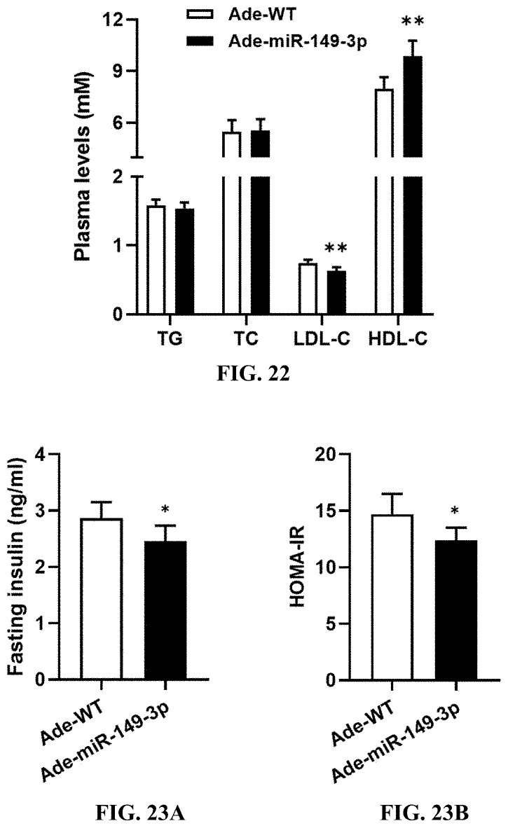

The plasma was tested for levels of TG, LC, LDL-C, and HDL-C. As shown in FIG. 22, when compared to the wild-type mice, the miR-149-3p-KO mice had comparable TG and LC levels, a lower LDL-C level, and a higher HDL-C level. The results indicated that miR-149-3p overexpression improved lipid disorders in the mice.

### 8. miR-149-3p Overexpression Increased Sensitivity of Mice to Insulin

The plasma was tested for insulin level. HOMA-IR was calculated from the fasting glucose levels. Fasting insulin (FIG. 23A) and HOMA-IR (FIG. 23B) levels were lower in the miR-149-3p-KO overexpression mice compared to the wild-type mice, indicating that miR-149-3p overexpression improved insulin sensitivity of the mice with insulin resistance.

### EXAMPLE 10

### miR-149-3p-KO Overexpression Activates Insulin Signaling Pathway

### 1. Virus Infection of Cells

Hepa1-6 cells were seeded into a 6-well plate, cultured overnight to a density of about 50%, and infected with virus. Adenovirus overexpressing miR-149-3p and control adenovirus were diluted by a serum-free cell culture medium to a concentration of 1×10⁸ cfu/mL. The Hepa1-6 cells were transferred from the serum-free cell culture medium to a serum-free medium. 20 μL of a diluted virus solution was added, mixed and cultured at 37° C. and 5% CO₂ for 12 hours. The Hepa1-6 cells were transferred into a complete medium and cultured for 48 hours. The cell pellets were collected for total RNA extraction.

### 2. Western Blot

The cell pellets were washed with PBS, broken with a lysis buffer (Beyotime, China), placed in a 4° C. ice bath for 30 minutes, and centrifuged at 13200 rpm for 15 minutes. The supernatant was collected as a protein sample and tested for protein concentration using BCA method (Multi Science, China). Proteins were separated by sodium dodecyl-sulfate polyacrylamide gel electrophoresis (SDS-PAGE) and transferred from a gel onto a nitrocellulose membrane. The nitrocellulose membrane was blocked with a 5% non-fat dry milk for 1 hour, washed three times with 1×TBST solution, and incubated overnight in a primary antibody at 4° C. The primary antibody (CST, USA) contained anti-phosphorylated AKT antibody (p-AKT), anti-total AKT antibody (t-AKT), phosphorylated GSK3β antibody (p-GSK3β), anti-total GSK3β antibody (t-GSK3β) and anti-GAPDH antibodies. The next day, the nitrocellulose membrane was removed from the primary antibody, washed three times, incubated in a secondary antibody (Proteintech, China) at room temperature for 1 hour, and washed three times. A luminescent solution (Thermo, USA) was evenly dropped on the nitrocellulose membrane and placed in a chemiluminescence imaging system.

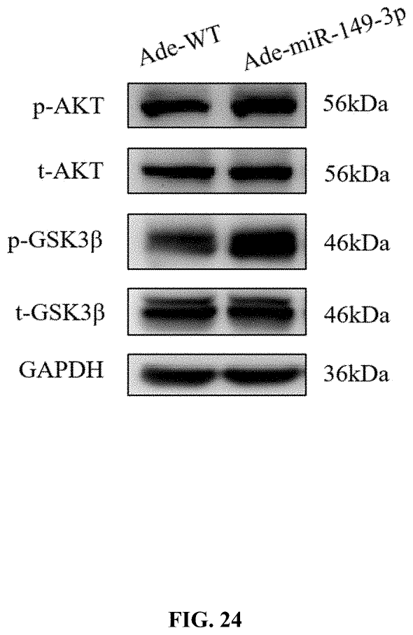

When an insulin signal activates phosphorylation of AKT in hepatocytes and skeletal muscle cells in vivo, GSK3 was phosphorylated and thus inactivated, and glycogen synthase was activated to promote glycogen synthesis, thus lowering blood glucose levels. Therefore, increases in the expression levels of phosphorylation of AKT protein (p-AKT) and GSK3β protein (p-GSK3β) were important indicators that the insulin signaling pathway had been activated. As shown in FIG. 24, when hepa1-6 cells are infected with adenovirus overexpressing miR-149-3p and control adenovirus, phosphorylation levels of p-AKT and p-GSK3β in the miR-149-3p overexpression cells are higher than in the control group. The results indicated that miR-149-3p overexpression in vitro activated the insulin signaling pathway.

Statistical analysis: all data were averaged from three independent repeated experiments, and the standard deviation (SD) was calculated and analyzed using the method in GraphPad Prism 8.0.1; P<0.05 was considered statistically significant, where *P<0.05; *P<0.01; ***P<0.001.

It will be obvious to those skilled in the art that changes and modifications may be made, and therefore, the aim in the appended claims is to cover all such changes and modifications.

What is claimed is:

1. MicroRNA, comprising one of or a combination of the following:
(a) a pri-miRNA of miR-149-3p;
(b) a pre-miRNA of miR-149-3p;
(c) a mature miRNA of miR-149-3p;
(d) a miR-149-3p derivative;
(e) a 18-26 nucleotides miRNA comprising a sequence of 5′-AGGGAGG-3′; and
(f) a derivative of the 18-26 nucleotides miRNA of (e).

2. The microRNA of claim 1, wherein the derivative in (d) and/or (f) is a cholesterol modifier, a locked nucleic acid modifier, a nucleotide modifier, a glycosylation modifier, a hydrocarbon modifier, a nucleic acid modifier, or a combination thereof.

3. The microRNA of claim 1, wherein in (e), the sequence of 5′-AGGGAGG-3′ is located in positions 2-8 of the miRNA; and the 18-26 nucleotides miRNA comprises more than 50% of activities of the miR-149-3p.

4. The microRNA of claim 1, wherein the mature miRNA of miR-149-3p comprises a RNA sequence represented by SEQ ID NO: 1, or a derivative thereof, and a DNA sequence encoding the mature miRNA is represented by SEQ ID NO: 2, or a derivative thereof.

5. A method for treating a metabolic disease, the method comprising: administering to a patient in need thereof an effective amount of a pharmaceutical composition comprising a mimic of miR-149-3p, or an expression vector of the miR-149-3p, wherein the metabolic disease is obesity, fatty liver, hyperlipidemia, diabetes, or atherosclerosis.

6. The method of claim 5, wherein the method further comprises integrating a DNA sequence encoding a mature miRNA of miR-149-3p to a viral vector or a eukaryotic vector to construct the expression vector of the miR-149-3p.

7. The method of claim 6, wherein the viral vector comprises an adenovirus vector, an adeno-associated virus vector, a retroviral vector, a herpes virus vector, or a combination thereof.

8. The method of claim 6, wherein the eukaryotic expression vector comprises is PCMV-myc expression vector, pcDNA3.0, pcDNA3.1, a modifier thereof, or a combination thereof.

9. The method of claim 5, wherein the composition further comprises a promoter, or an enhancer.

10. The method of claim 5, wherein the pharmaceutical composition comprising the expression vector of the miR-149-3p is introduced or transfected into the patient's cells or allogeneic cells in vitro, and the cells are amplified in vitro and then transferred to the patient.

11. The method of claim 5, wherein the pharmaceutical composition comprising the mimic of miR-149-3p, or the expression vector of the miR-149-3p is directly introduced to the patient.

12. The method of claim 5, wherein the diabetes is type 2 diabetes.

13. The method of claim 5, wherein the mimic of miR-149-3p is a cholesterol modified miR-149-3p mimic.
